(19) United States
(12) Patent Application Publication
(10) Pub. No.: US 2008/0154233 A1
(43) Pub. Date: Jun. 26, 2008

(54) APPARATUS FOR DELIVERING A BIOCOMPATIBLE MATERIAL TO A SURGICAL SITE AND METHOD OF USING SAME

(75) Inventors: Jian Q. Yao, Austin, TX (US); Victor Zaporojan, Austin, TX (US); Jizong Gao, Cedar Park, TX (US); Xiao Huang, Austin, TX (US); Joe Zhe Guo, Austin, TX (US)

Correspondence Address: WOOD, HERRON & EVANS, LLP (ZIMMER), 2700 CAREW TOWER, 441 VINE STREET, CINCINNATI, OH, 45202, US

(73) Assignee: ZIMMER ORTHOBIOLOGICS, INC., Austin, TX (US)

(21) Appl. No.: 11/613,319

(22) Filed: Dec. 20, 2006

(51) Int. Cl. A61M 5/00; A61M 31/00

(52) U.S. Cl. 604/506; 604/264

(57) ABSTRACT

A device for delivering a biocompatible material to a surgical site includes an elongate cannula having a proximal portion, a distal portion, and at least one interior lumen disposed between the proximal and distal portions through which the biocompatible material is delivered. The device further includes a confinement member adjacent the distal portion of the cannula having expanded and unexpanded positions. In the expanded position, the confinement member defines an open cavity about the distal portion of the cannula and in fluid communication with the interior lumen to retain the biocompatible material. A method of delivering a curable biocompatible material to a surgical site includes positioning a distal portion of a cannula adjacent the surgical site and actuating a confinement member to define an open cavity about the distal portion. The biocompatible material is introduced into the cavity through the cannula and cross-linking of the curable biocompatible material is initiated.

### FIELD OF THE INVENTION

An apparatus and method for surgical procedures, more particularly a minimally invasive apparatus and method for delivering a biocompatible material to a surgical site during various orthopedic procedures.

### BACKGROUND OF THE INVENTION

The musculoskeletal system is subject to injury caused by traumatic events as well as by a number of diseases. Repair of connective tissue of the musculoskeletal system is commonly performed. By way of example, articular cartilage is a type of hyaline cartilage that lines the surfaces of the opposing bones in a diarthrodial joint (e.g., knee, hip, shoulder, etc.). Its primary function is to permit smooth, near frictionless movement during articulation between bones of the joint by providing a low-friction interface between the contacting cartilage surfaces of the joint. Articular cartilage is also load bearing, and serves to transmit and distribute compressive joint loads to the underlying subchondral bone.

Articular cartilage is typically damaged in one of two ways, acute trauma suffered through physical activity (such as twisting motion of the leg, sharp lateral motion of the knee, or repetitive impact), or degenerative conditions (such as arthritis or systemic conditions). In addition, as a person ages, articular cartilage loses mechanical strength, rendering the cartilage even more susceptible to trauma. Because articular cartilage tissue is aneural, i.e., having few or no nerves, and avascular, i.e., having few or no blood vessels, the healing of damaged cartilage is limited.

Consequently, various surgical methods are available for the treatment of damaged tissue, such as cartilage. In one treatment approach, the damaged tissue is removed and replaced with natural or synthetic materials that are physiologically acceptable to the human body and which perform the function formerly performed by the material removed. Recently, various orthopedic surgical procedures have replaced native tissue, such as cartilage, with a curable biocompatible material. Such surgical procedures have been performed using minimally invasive techniques, such as arthroscopic and endoscopic techniques, that allow as much of the healthy tissue as possible to remain. Biocompatible materials that have shown promise for effecting soft tissue repair are hydrogels. Hydrogels are particularly suitable for minimally invasive procedures because they provide controllable phase change, such that the hydrogel may be injected through the minimally invasive device while in a liquid state and then cured in-situ to form a solid or a gel.

While the use of hydrogels has generally been successful to effect joint repair, their use does have some drawbacks. One such drawback is that while the hydrogel is in a liquid form, such as when delivering the hydrogel to a surgical site through the minimally invasive device, it has a relatively low viscosity. Consequently, the hydrogel flows easily and is therefore difficult to contain at the treatment site. Moreover, leakage of the hydrogel onto and/or into the tissue surrounding the surgical site may not be desirable in some surgical procedures. As a result, the use of hydrogels to effect joint repair has been heretofore limited.

Therefore, there is a need for improvements in a method and apparatus for delivering a biocompatible material to a surgical site.

### SUMMARY OF THE INVENTION

Apparatus and method of delivering a biocompatible material to a surgical site that confines the biocompatible material to a desired target area of the surgical site. The apparatus and method may also reduce or prevent the leakage of the biocompatible material to the surrounding tissue.

In one embodiment, a device for delivering a biocompatible material to a surgical site during a surgical procedure, such as a minimally invasive surgical procedure, includes an elongate cannula having a proximal portion adapted to be located outside a body during the surgical procedure and a distal portion adapted to be located within the body during the surgical procedure and adjacent the surgical site. The elongate cannula includes at least one interior lumen disposed between the proximal and distal portions through which the biocompatible material is delivered. The device further includes a confinement member adjacent the distal portion of the cannula having an unexpanded position and an expanded position. When the confinement member is in the unexpanded position, the cannula is adapted to be inserted into the body and moved toward the surgical site. Once adjacent the surgical site, however, the confinement member may be deployed in its expanded position. In the expanded position, the confinement member defines an open cavity about the distal portion of the cannula which is in fluid communication with the at least one interior lumen to retain at least a portion of the biocompatible material.

In one exemplary embodiment, the confinement member may be an inflatable member that may be pneumatically actuated to move to the expanded position and deflated to move to the unexpanded position. The confinement member may also be hydraulically or mechanically actuated to move between the expanded and unexpanded position. Additionally, the confinement member may be integrally formed with the cannula and have a cone shape when in the expanded position. A vacuum source may be coupled to the at least one lumen for aspirating fluid from the cavity defined by the confinement member. The confinement member may include a ring member that facilitates sealing of the distal portion of the confinement member with the body tissue surrounding the defect site. In another embodiment, the distal portion of the confinement member may include a membrane that closes the opening to the cavity. The membrane may include an aperture, such as an aperture defined by a luer lock connector, to provide fluid communication between the cavity and the defect site.

Once the biocompatible material is delivered to the cavity of the confinement member, cross-linking may be initiated to cure the biocompatible material in-situ. For example, in one embodiment, the delivery device may include a light source for photo initiating the cross-linking of the curable biocompatible material. The light from the light source may be introduced into the cavity through the cannula itself or may be introduced from a source external to the cannula. For example, a second cannula inserted adjacent the surgical site may provide the light for photo initiating the biocompatible material. To this end, at least a portion of the confinement member is capable of transmitting light therethrough to photo initiate the biocompatible material in the cavity. In one embodiment, the light source may be visible light and/or ultraviolet light. In addition, at least a portion of confinement member may be formed of a transparent material to provide visualization of the surgical site through the confinement member.

A method of delivering a curable biocompatible material to a surgical site in the body includes positioning a distal portion of a cannula adjacent the surgical site and actuating a confinement member to define an open cavity about the distal portion of the cannula. The curable biocompatible material is introduced into the cavity through the cannula and cross-linking of the curable biocompatible material is initiated. Actuating the confinement member may include inflating the confinement member to an expanded position. Initiating cross-linking of the biocompatible material may be accomplished with at least one of photo initiation, thermal initiation, or chemical initiation. In one embodiment, initiation is through photo initiation using visible and/or ultraviolet light. In one embodiment, light passes through at least a portion of the confinement member to photo initiate the cross-linking of the curable biocompatible material in the cavity. The method may further include visualizing the surgical site through at least a portion of the confinement member. Furthermore, vacuum pressure may be used to aspirate fluid from the cavity defined by the confinement member. Once the biocompatible material has at least partially cured, the confinement member may be moved to an unexpanded position, such as by deflation, and the cannula removed from the surgical site.

These and other embodiments will become more readily apparent to those of ordinary skill in the art upon review of the following detailed description taken in conjunction with the accompanying drawing.

### BRIEF DESCRIPTION OF THE DRAWING

The accompanying drawings, which are incorporated in and constitute a part of this specification, illustrate embodiments of the invention and, together with a general description of the invention given above, and the detailed description given below, serves to explain the invention.

FIG. 1 is a cross-sectional view of an apparatus for delivering a biocompatible material to a surgical site in accordance with an embodiment of the invention;

FIG. 2 is a cross-sectional view of an apparatus for delivering a biocompatible material to a surgical site in accordance with another embodiment of the invention; and

FIG. 3 is a cross-sectional view of an apparatus for delivering a biocompatible material to a surgical site in accordance with yet another embodiment of the invention.

### DETAILED DESCRIPTION

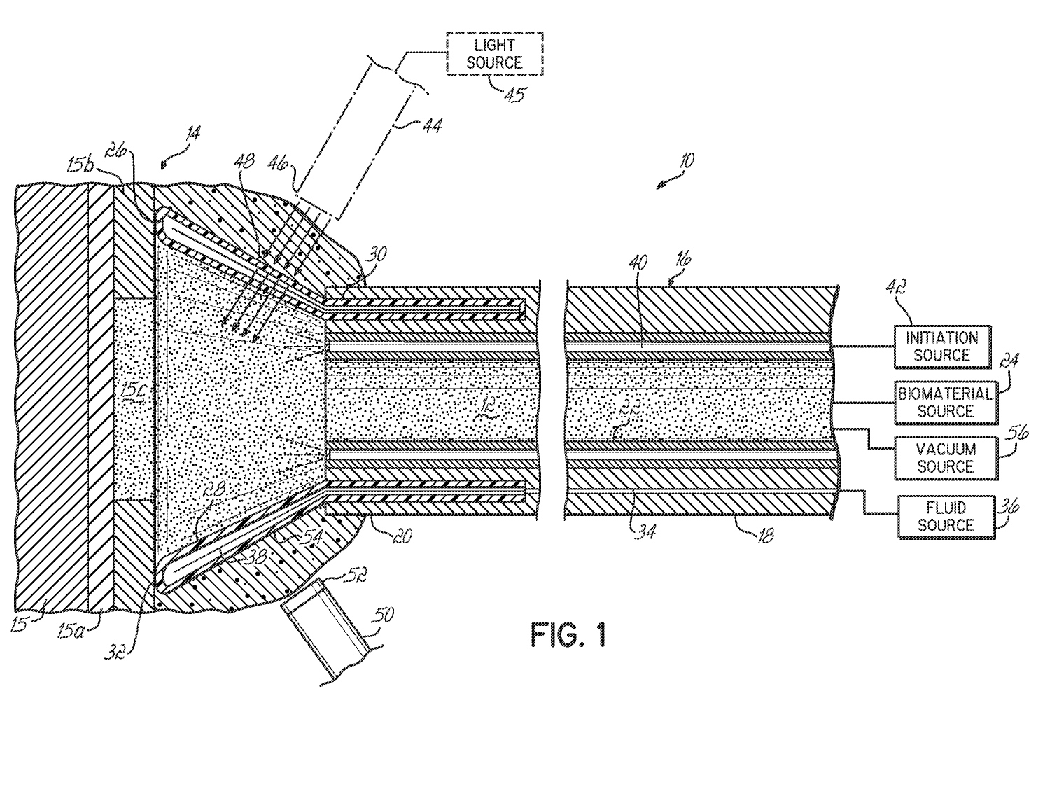

Referring to FIG. 1, a device 10 for delivering a biocompatible material 12 to a surgical site 14 is schematically illustrated. The device 10 may be used in various surgical procedures, including orthopedic surgical procedures to effect repair of the musculoskeletal system. In an exemplary embodiment, the device 10 may be used in minimally invasive procedures, such as arthroscopic and endoscopic procedures, to effect repair of a joint. In one embodiment, the device 10 may be used to repair the cartilage within a diarthrodial joint, such as the knee. By way of example, as shown in FIG. 1, the surgical site 14 may include bone 15, subchondral bone 15a, and cartilage 15b wherein cartilage 15b includes a defect 15c that is to be repaired using embodiments of the invention. The invention, however, is not so limited as those of ordinary skill in the art will recognize a wide range of surgical applications that may benefit from embodiments of the invention described herein. Thus, embodiments of the invention are not to be limited to orthopedic surgical procedures in general, or to the repair of cartilage in diarthrodial joints in specific.

The device 10 includes an elongate cannula 16 having a proximal portion 18 located outside the body of a patient during a surgical procedure, and a distal portion 20 located within the body of the patient and proximate the surgical site 14. The device 10 includes at least one interior lumen 22 disposed between the proximal and distal portions 18, 20 through which the biocompatible material 12 is delivered. The device 10 may include a supply or reservoir 24 of biocompatible material in fluid communication with the proximal portion 18 of the cannula 16 to supply the interior lumen 22 with the biocompatible material 12.

For minimally invasive procedures, controllable phase change of the biocompatible material may facilitate the delivery of the biocompatible material 12 to the surgical site 14. In particular, in one exemplary embodiment, the biocompatible material 12 may be delivered to the surgical site 14 while in a liquid state and then subsequently cured in-situ to form a solid or gel. The liquid state of the biocompatible material 12 allows that material to flow through the interior lumen 22 of the cannula 16 and be delivered to the surgical site 14 without the need for more invasive surgical techniques that may typically be used for locating the biocompatible material 12 at the surgical site 14.

A hydrogel is one such biocompatible material 12 that can exhibit such phase change properties, i.e., may be delivered in a liquid state and then subsequently cured in-situ, and which may be used in the invention. Cured hydrogels may exhibit physical/chemical characteristics analogous to those of human soft tissue, such as cartilage, and can demonstrate a combination of such properties as load bearing, shear stress resistance, impact absorption, and/or wear characteristics. The term hydrogel includes liquid and/or semi-solid long chain hydrophilic molecules that form cavities or spaces that contain entrapped liquids, typically water, within the cavities at a concentration ranging from about 20% to about 95%. The cavities absorb water (or other liquids) from the surrounding environment, and can slowly release the water as the molecules biodegrade.

Hydrogels may be classified according to composition (homopolymer, copolymer, multipolymer, or interpenetrating hydrogels), ionic charge (neutral, anionic, cationic, or ampholytic hydrogels), and/or structure (amorphous, semicrystalline, or hydrogen-bonded hydrogels). Methods, components, concentrations, conditions, etc. to produce hydrogels are known by one skilled in the art such as described in U.S. Pat. Nos. 6,949,590; 6,511,650; 6,497,902; Published U.S. Patent Application No. 20060252159; and Hoffman (Advanced Drug Delivery Reviews, Vol. 43, 2002, pp 3-12) each of which are incorporated herein by reference in its entirety.

Hydrogels may be prepared from natural polymers that include, but are not limited to, collagen, hyaluronate, chitosan, gelatin, algenate, pectin, carrageenen, chondroiten sulfate, dextran sulfate, polylysine, carboxymethyl chitin, fibrin, dextran, agarose, and pullulan. Hydrogels may be prepared from synthetic polymers that include, but are not limited to, poly(2-hydroxyethylmethacrylate (HEMA), polyphazene, poly(ethylene oxide) PEO and its copolymers, polyesters such as PEG (polyethylene glycol)-PLA (polylactic acid)-PEG, PEG-PLGA-PEG, PEG-PCL (polycaprolactone)-PEG, PLA-PEG-PLA, PHB (poly(3-hydroxybutyrate)), P(PF-co-EG) plus or minus acrylate end groups, P(PER/PBO terephthalate), other polymers such as PEG-bis-PLA-acrylate), PEG-g-P(Aam-co-Vamine), PAAm, P(NIPAAm-co-Aac), P(NIPAAm-co-EMA), PVAc/PVA, PNVP, P(MMA-co-HEMA), P(AN-co-allyl sulfonate), P(biscarboxy-phenoxy-phosphazine), P(GEMA-sulfate). Hydrogels may be prepared from both natural and synthetic polymers, examples of which include, but are not limited to, P(PEG-co-peptides), alginate-g-(PEO-PPO-PEO), P(PLGA-co-serine), collagen-acrylate, alginate-acrylate, P(HPMA-g-peptide), P(hema/Matrigel®), and HA-g-NIPAAm.

Hydrogels may be prepared from branched deoxyribonucleic acid (DNA) that self-forms into various shapes (e.g., a cross, a “Y”, a “T”). These may have non-base paired termini to which a complementary sequence may anneal (i.e., “sticky ends”). These may be used with ligases to link DNA strands to each (e.g., Steele, B. Sep. 28, 2006, Cornell CHONICLE, page 7). Cross-shaped branched DNA forms a gel by linking into sheets of tiny squares that tangle in three dimensions; Y shapes form hexagonal structures like a chain link fence that combine into a fibrous three-dimensional form; T shapes create random, disorganized patterns that resemble scales, etc. Properties such as rigidity and/or absorbance of the resulting hydrogels may be altered by adjusting the types of branched DNA used and the DNA concentration.

In one embodiment, hydrogels are long-chain molecules cross-linked to one another. In another embodiment, hydrogels are long-chain molecules that are not cross-linked; while these are able to absorb liquids within their cavities, they do not biodegrade (i.e., they do not dissolve) due to the presence of hydrophobic and hydrophilic regions in their structure. The term hydrogel is also applied to hydrophilic polymers in a dry state (xerogel).

Cross-linking may be effected by physical, chemical, and/or photo cross-linking. Physical cross-linking occurs due to ionic linkages, hydrogen bonding, van der Waals forces, or other physical forces. Chemical cross-linking occurs due to formation of covalent linkages using chemical initiators. Photo cross-linking, also termed photopolymerization, of hydrogels may occur by exposure to ultraviolet and/or visible light, either in the presence or absence of a photo initiator. Examples of polymers and methods of use are described in U.S. Pat. Nos. 5,567,435 and 6,156,478; and Published U.S. Patent Application No. 20060252159. Examples of polymers/monomers suitable to form ionically cross-linked hydrogels with adjustable gellation times are disclosed in U.S. Pat. No. 6,497,902. Examples of polymers suitable to form porous hydrogels are disclosed in U.S. Pat. No. 6,511,650. Examples of polymers suitable to form bioabsorbable polymer hydrogels for sustained release of drugs are disclosed in Published U.S. Patent Application No. 2006/0251719.

Hydrogels may contain both hydrophobic and hydrophilic components. Preparation of these hydrogels does not rely on use of copolymers or physical blending, but instead relies on hydrophobic and hydrophilic components. These components are convertible into a one phase cross-linked polymer network structure by free radical polymerization, as described in U.S. Patent Application Publication No. U.S. 2002/0161169.

Hydrogels may be formulated as temperature sensitive compounds, described in U.S. Patent Application Publication No. U.S. 2006/0188583. Polymers, either commercially available or synthesized, are dissolved in water or other liquid, and an agent that facilitates cross-linking such as sodium hyaluronate (SH) is added. The temperature sensitive hydrogel are liquid at about ambient room temperature (about 20° C.) and transition to become a solid (gel) at about body temperature (about 37° C.). Any polymers may be used to prepare temperature sensitive hydrogels as long as it possess the necessary properties to support the hydrogel. Examples of such polymers include, but are not limited to, N-isopropyl acrylamide polymer, ethylhydroxyethylcellulose and its derivatives, poly(ethylene glycol)/poly(D,L-lactic acid-co-glycolic acid) block co-polymers and analogs, and poly(etheylene oxide-b-propylene oxide-b-ethylene oxide) (Poloxamers or PLURONICS® polymers, which are block copolymers of the type ABA, consisting of a central, hydrophobic block of polypropylene oxide, which is edged by two hydrophilic blocks of polyethylene oxide. The polymers are derived from the sequential polymerization of propylene oxide and ethylene oxide).

Hydrogels may be polymerized in-situ. U.S. Published Patent Application No. 2006/018894 describes in-situ polymerization of a hydrogel using UV light in the presence of stratum corneum tissue. U.S. Published Patent Application No. 2004/0241203 describes a fluid composition comprising particulate material, and a cross-linking agent, the particles cross-linking to form a matrix on introduction to the cross-linking agent in or on a target tissue. Changing the amount of monomer and cross-linker can change the thickness and pore size of hydrogel layers as described in PCT application WO 00/66265.

Hydrogels may serve as an extracellular matrix (ECM), or bioscaffold, to provide a surface upon which cells can attach. This may have applications in tissue engineering implantation. As one example, Schmedlen et al (Biomaterials 23 (2002) 4325) describe polyvinyl alcohol hydrogels that can be modified with cell adhesion peptides. As another example, Khademhosseini et al. describe gradient hydrogels embedded with the peptide Arg-Gly-Asp (RGD) that can bind cell integrins (membrane bound receptors). Published U.S. Patent Application No. 2006/0233850 discloses bioscaffolds formed of hydrogels that are cross-linked in-situ in an infarcted region of the heart.

Hydrogels may serve as drug delivery devices. In one embodiment, a hydrogel may gradually dispense a drug or other liquid within its cavities (e.g., U.S. Patent Application Publication No. 2006/0251719 discloses a sustained-release, bioabsorbable polymer hydrogel drug preparation). Such hydrogels form a complex with the drug through physiochemical interactions to effect sustained drug release, in effect forming a microcapsule. Techniques for preparing, loading, etc. such hydrogels are known to one skilled in the art.

The invention, however, is not limited to hydrogels, as those of ordinary skill in the art will recognize other suitable biocompatible materials capable of being delivered to the surgical site by means of a cannula, and cured in-situ to form a replacement material during a surgical procedure.

As noted above, however, the biocompatible material 12, such as a hydrogel, may exhibit a relatively low viscosity when in the liquid state. The biocompatible material 12 then flows easily and thus passes through the cannula 16 with reduced resistance to flow and with a relatively small pressure gradient. While this may be desirable to facilitate delivery of the biocompatible material 12 through the device 10, the relatively low viscosity may make confining the biocompatible material 12 to a desired target area of the surgical site 14 challenging. In other words, the enhanced flowability of the biocompatible material 12 may allow the material to essentially leak into or onto the tissue surrounding the surgical site 14 or other areas where no biocompatible material is desired.

To address the flowability of the biocompatible material 12 at the surgical site 14, the device 10 may further include a confinement member 26 adjacent the distal portion 20 of the cannula 16. The confinement member 26 is adapted to at least partially bound or confine the biocompatible material 12 at the surgical site 14 so as to reduce or prevent leakage of biocompatible material beyond the desired target area. The confinement member 26 has an unexpanded position and an expanded position. In the unexpanded position, the confinement member 26 may be retracted or has a relatively small or reduced size so as to facilitate the movement of the cannula 16 toward the surgical site 14 and through the body tissue, as is typical in minimally invasive procedures.

In the expanded position, however, the confinement member 26 defines a cavity 28 about the distal portion 20 of the cannula 16. A proximal portion 30 of the confinement member 26 is coupled to the distal portion 20 of the cannula 16 and a distal portion 32 of the confinement member 26 projects beyond the distal portion 20 of the cannula 16. In one embodiment, the distal portion 32 of the confinement member 26 is open such that cavity 28 is an open cavity. Moreover, as shown in FIG. 1, the cavity 28 is in fluid communication with the interior lumen 22 for delivering the biocompatible material 12 to the cavity 28, as explained in more detail below.

In one embodiment, the confinement member 26 may be configured as a balloon-type member capable of being inflated and deflated so as to define the unexpanded and expanded positions. More particularly, the confinement member 26 may be fluidicly actuated so as to move between the unexpanded and expanded positions. To this end, the elongate cannula 16 may include a lumen 34 in fluid communication with the confinement member 26 adjacent the distal portion 20 of the cannula 16 and in fluid communication with a fluid source 36 (e.g., a gas or liquid) adjacent the proximal portion 18 of the cannula 16 to pneumatically or hydraulically expand the confinement member 26. A controller (not shown) may be operatively coupled to the device 10 and/or fluid source 36 to control the movement of the confinement member 26 between the unexpanded and expanded positions. In one embodiment, the confinement member 26 is integrally formed with the cannula 16. In another embodiment (not shown), the confinement member 26 may be separately coupled to the distal portion 20 of cannula 16.

In one exemplary embodiment, the confinement member 26 may be cone shaped so as to provide a cavity 28 larger than the distal portion 20 of the cannula 16. In one embodiment, the confinement member 26 may further include a reinforcing material 38 (e.g., a mesh, fibers, ribs, grooves, or other reinforcing structures) embedded within the material that forms the confinement member 26 or otherwise coupled to or bounded by the material that forms the confinement member 26. The reinforcing material 38 provides increased strength and rigidity to the confinement member 26 but does not otherwise unduly hinder movement of the confinement member 26 between the unexpanded and expanded positions. This reinforcing material 38 provides increased support to the confinement member 26 so that it retains the desired shape at the surgical site 14, e.g., a cone. The reinforcing material 38 may further apply a bias to the confinement member 26 so that as it moves toward the expanded position, such as during pneumatic or hydraulic actuation, the confinement member 26 takes the desired shape. While the confinement member 26 is shown and described as having a cone shape, the invention is not so limited. As those of ordinary skill in the art will recognize, depending on the particular application, the confinement member 26 may have a wide variety of shapes and sizes, including irregular shapes. For example, the confinement member 26 may be adapted to form shapes in the expanded position, including without limitation: rectangular, cylindrical, conical, oval, square, polygonal, multi-lobed, star-shaped, etc. In such applications, the confinement member 26 may be configured so as to conform to such an irregular shape when in the expanded position. The invention, therefore, is not limited to the cone shape shown and described herein, but may have a wide variety of shapes and sizes to accommodate the particular application. In addition, the cavity 28 my have a wide variety of shapes and sizes depending on the specific application. The cavity 28 may have a shape corresponding to the shape of the confinement member 26. For example, as shown in FIG. 1, both the confinement member 26 and cavity 28 have a conical shape. The invention, however, is not so limited as the shape of cavity 28 may be different than the shape of the confinement member 26. Thus, the cavity 28 may be adapted to form shapes including without limitation: rectangular, cylindrical, conical, oval, square, polygonal, multi-lobed, star-shaped, etc., irrespective of the shape of the confinement member 26.

In addition, the confinement member 26 may also be moved between the unexpanded and expanded positions using means other than pneumatic or hydraulic actuation. For instance, the confinement member 26 may include or be formed from a flexible shape memory alloy that is movable between an unexpanded position within the cannula 16 and an expanded position external to the cannula 16. When external to the cannula 16, the flexible shape memory alloy is permitted to deform to a pre-determined configuration that is desired for the particular application. Alternately, the confinement member 26 may be made from a material responsive to temperature, such that at one temperature the confinement member 26 is in an unexpanded position, and at a second temperature, the confinement member 26 is in an expanded position. Examples of such materials include without limitation shape memory thermoplastics including polyetherurethane (TFX) and biodegradable multiblock copolymer (PDC). Furthermore, the confinement member 26 may be mechanically actuated to move between the unexpanded and expanded positions. For example, a wire or other linking element (not shown) may extend between the confinement member 26 and the proximal portion 18 of the cannula 16 located outside the body. The wire may be coupled to a plurality of ribs (not shown) such that movement of the wire causes the ribs to move the confinement member 26 between the expanded and unexpanded positions. Such a configuration would permit the confinement member 26 to operate in an umbrella-like fashion. Those of ordinary skill in the art will recognize other confinement member configurations that move between unexpanded and expanded positions within the scope of the invention.

Once the biocompatible material 12 has been delivered to the surgical site 14 it may be cured in-situ. To this end, cross-linking of the biocompatible material 12 may be initiated. Cross-linkable solutions used to form biocompatible materials, such as hydrogels, include those that form physical cross-links, chemical cross-links, or both. Physical cross-links may result from complexation, hydrogen bonding, desolvation, van der Waals interactions, ionic bonding, etc., and may be initiated by mixing two components that are physically separated until combined in-situ, or as a consequence of a prevelant condition in the physiological environment, such as temperature, pH, ionic strength, light, etc. Chemical cross-linking may be accomplished by any of a number of mechanisms, including free radical polymerization, condensation polymerization, anionic or cationic polymerization, step growth polymerization, etc.

In embodiments of the invention, the biocompatible material 12 may be initiated in several ways to cause curing, including photo initiation, thermal initiation and/or chemical initiation. In particular, in an exemplary embodiment of the invention, cross-linking of the biocompatible material 12 may be initiated using photo initiation. To this end, the cannula 16 may include an initiation lumen 40 that facilitates initiation of cross-linking of the biocompatible material 12 from an initiation source 42 operatively coupled to the device 10. In one embodiment, the lumen 40 may be a light lumen capable of passing light therethrough from an initiation source 42 configured as a light source. In such an embodiment, the initiation source 42 may be a fiber optic bundle positioned within the cannula 16 itself, such that the light source is adjacent the distal portion 20 of the cannula 16, or the light source may be positioned away from the surgical site 14 whose light is simply channeled through the lumen 40. For example, a surgical instrument (forceps, scalpel, etc.) may contain or be configured with an illumination system. Such instruments are known to one skilled in the art, such as fiber optic instruments available from BioSpec (Moscow, Russia) for example, TC-1 fiber optic tool for photodynamic therapy with fine needle tip (www.biospec.rv/Fiber_Optics_e.html). In any event, light from the initiation lumen 40 is transmitted into the cavity 28 of the confinement member 26 to initiate curing of the biocompatible material 12 therein. The biocompatible material 12 may be configured such that visible light and/or ultraviolet light initiates cross-linking. Accordingly, the light source may be configured to generate visible and/or ultraviolet light as required by the specific application or cross-linking system.

In alternate embodiments of the invention, light that causes cross-linking of the biocompatible material 12 in cavity 28 may be introduced in ways other than through the cannula 16 itself. By way of example, and as shown in phantom in FIG. 1, the device 10 may include a second cannula 44 that is inserted into the body of the patient such that its distal portion 46 is adjacent the surgical site 14. The second cannula 44 is coupled to a light source 45 and is capable of passing light out of the distal portion 46 of cannula 44. Moreover, at least a portion 48 of the confinement member 26 is capable of transmitting light therethrough. In this way, light from second cannula 44 may pass through the confinement member 26 and photo initiate the biocompatible material 12 that has been delivered to cavity 26 through cannula 16. For example, portion 48 of confinement member 26 may be formed of polyurethane or other suitable materials that allow light to pass therethrough. The light from second cannula 44 may be visible and/or ultraviolet light, as dictated by the specific application or cross-linking system.

As noted above, the biocompatible material 12 may be initiated by means other than photo initiation. To this end, the initiation source 42 may alternatively be configured as a heating wire or other heat generating element (not shown) that is capable of generating heat sufficient to initiate cross-linking of the biocompatible material 12 in cavity 28 via thermal initiation. The heating wire may be located within initiation lumen 40 and adjacent distal portion 20 or the heating wire may be remote from the surgical site 14 and the heat transferred to the biocompatible material 12 in cavity 28 via the lumen 40. In yet another alternate embodiment, the initiation source 42 may be configured as a reservoir or source of a chemical agent (not shown) that may be combined with biocompatible material 12 in cavity 28 to initiate cross-linking via chemical initiation. In such an embodiment, the initiation lumen 40 provides fluid communication between the reservoir of chemical agent and the biocompatible material 12 in cavity 28.

In other embodiments, the device 10 may further include yet a third cannula 50 inserted into the body of the patient such that its distal portion 52 is proximate the surgical site 14. The third cannula 50 carries optical instrumentation for viewing the surgical site 14, as is generally known in the art. In particular, the surgical site 14 may be viewed through the confinement member 26. To this end, at least a portion 54 of the confinement member 26 is formed from a transparent material that allows visualization of the surgical site 14 through the confinement member 26.

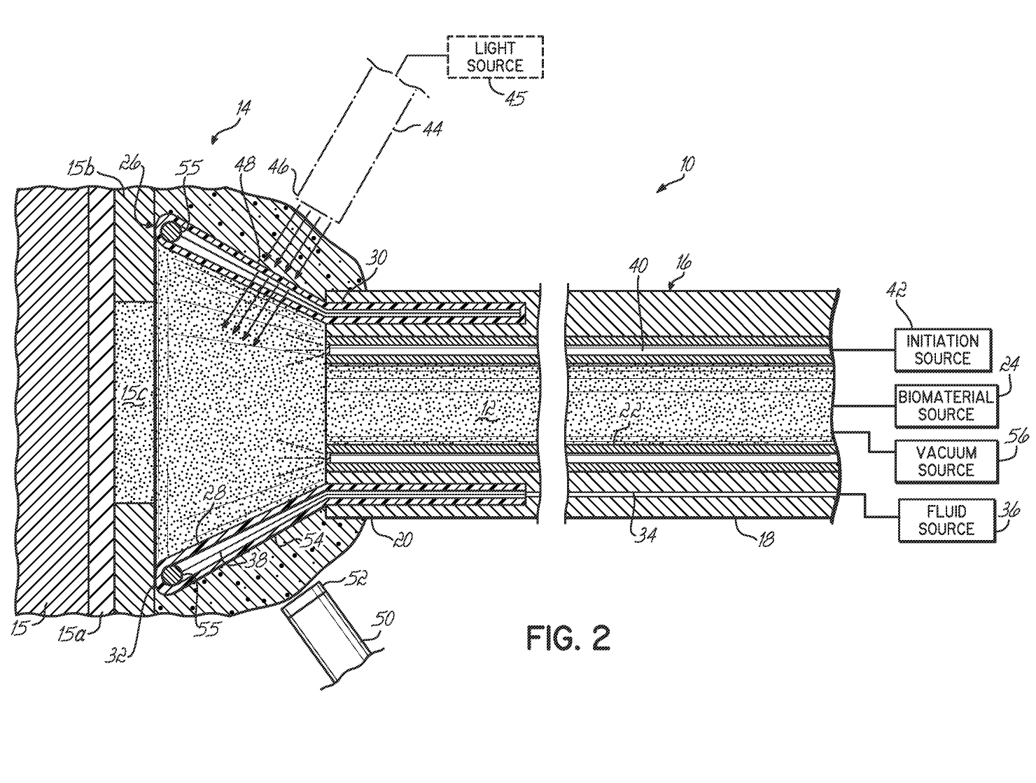

In use, the cannula 16 is inserted into the body of a patient and advanced so that its distal portion 20 is adjacent the surgical site 14. The confinement member 26 may be in its unexpanded state as the cannula 16 is being moved toward the surgical site 14. Once positioned, the confinement member 26 is actuated to its expanded position. For example, the confinement member 26 may be pneumatically, hydraulically or mechanically actuated, but is not limited to such an actuation. In the expanded position, the confinement member 26 defines the open cavity 28. The distal portion 32 of the confinement member 26 may be positioned against native tissue, such as bone, tendons, ligaments, healthy cartilage, etc, so as to essentially close off the opening to cavity 28. In other words, the confinement member 26 may form a seal with surrounding tissue to create a substantially closed cavity. As shown in FIG. 2, in which like reference numerals refer to like features in FIG. 1, the distal portion 32 of the confinement member 26 may include a ring member 55 that facilitates sealing of the confinement member 26 with the tissue at the surgical site 14. For example, the tissue surrounding defect 15c may not be smooth but may be rough or irregular. In these applications, the ring member 55 may supply a force that allows the distal portion 32 of the confinement member 26 to conform to the irregular contour of the tissue to promote sealing and thus preventing or reducing the leakage of the biocompatible material 12 outside the target area.

Before delivering the biocompatible material 12 to the cavity 28, it may be necessary or desirable to aspirate any fluid in cavity 28. To this end, the interior lumen 22 may be operatively coupled to a vacuum source 56 to aspirate any fluid in cavity 28. While the vacuum source 56 is shown as being operatively coupled to interior lumen 22, those of ordinary skill in the art will recognize that the fluid may be aspirated through other lumens in cannula 16, such as a lumen dedicated only to the aspiration of fluids. In addition, the interior lumen 22 may also be used to further prepare the surgical site 14. For example, in some applications, the interior lumen 22 may be used to deliver a fluid (e.g., saline solution, air, nitrogen or other gases, etc.) to lavage the surgical site 14 or to dry the surgical site 14. In other applications, the interior lumen 22 may be used to deliver instruments (e.g., scalpel, curettes, etc.) to the surgical site 14 to, for example, contour the underlying tissue. In still other applications, the interior lumen 22 may be used to deliver substances other than biocompatible material 12 to the surgical site 14. For example, hemostatic agents, antibiotics, etc. may be delivered to the surgical site 14 via interior lumen 22. Those of ordinary skill in the art will recognize that dedicated lumens in cannula 16 may be used instead of interior lumen 22.

With the surgical site 14 prepared, the biocompatible material 12 is delivered to the cavity 28 via the lumen 22 and at least partially retained therein by the confinement member 26. In this way, the flowable biocompatible material 12 does not undesirably leak into or onto the surrounding areas of the surgical site, but is confined or retained within a targeted area of the surgical site 14. Once the biocompatible material 12 is delivered to the cavity 28, in-situ cross-linking of the biocompatible material 12 may be initiated. For example, the cross-linking may be initiated by photo initiation, thermal initiation, and/or chemical initiation. In particularly preferred embodiments, the biocompatible material may be photo initiated.

Accordingly, visible and/or ultraviolet light from an initiation source 42 may be introduced into the cavity 28 either from the cannula 16 itself or from external to the cannula, such as from second cannula 44. In the embodiment for which light is introduced externally from the cannula 16, at least a portion of the confinement member 26 is capable of passing light therethrough so as to photo initiate the biocompatible material 12 in cavity 28. Furthermore, a third cannula 50 may be utilized to view the surgical site 14 through the confinement member 26. In this case, at least a portion 54 of the confinement member 26 is transparent to facilitate viewing of the surgical site 14. When the biocompatible material 12 has at least partially cured, the confinement member 26 may be returned back to its unexpanded state, such as by deflation, and the cannula 16 is removed from the body of the patient.

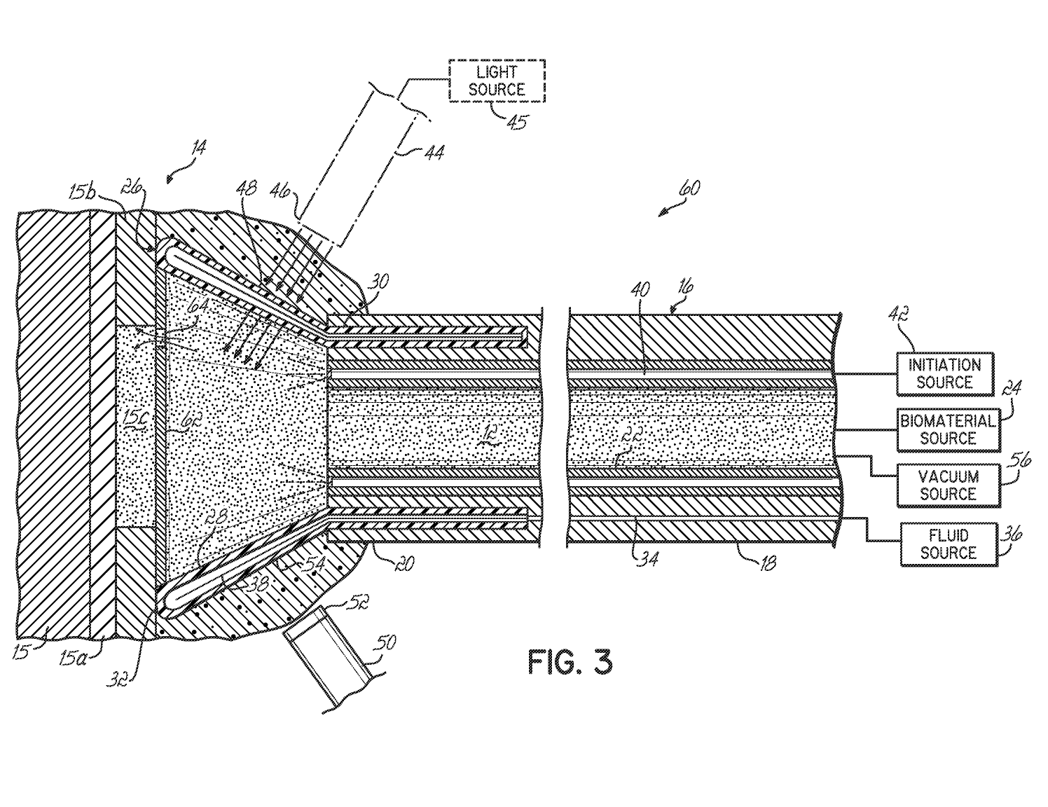

FIG. 3, in which like reference numerals refer to like features in FIG. 1, shows another embodiment of a device 60 for delivering biocompatible material 12 to surgical site 14. In this embodiment, however, the distal portion 32 of the confinement member 26 includes a membrane 62 that essentially closes off cavity 28. The membrane 62 further includes an aperture 64 that provides fluid communication between cavity 28 and the surgical site 14. For example, the aperture 64 may be positioned so that the cavity 28 is in fluid communication with defect 15c so that biocompatible material 12 may flow into defect 15c but be prevented from flowing into or onto the surrounding body tissue by confinement member 26. The aperture 64 may be defined, for example, by a luer lock connector or other suitable connector (not shown).

While the present invention has been illustrated by a description of various preferred embodiments and while these embodiments have been described in some detail, it is not the intention of the inventors to restrict or in any way limit the scope of the appended claims to such detail. Additional advantages and modifications will readily appear to those skilled in the art. The various features of the invention may be used alone or in numerous combinations depending on the needs and preferences of the user.

What is claimed is:

1. A device, comprising:
an elongate cannula having a proximal portion adapted to be located outside a body during a procedure, a distal portion adapted to be located within the body during the surgical procedure and adjacent a site to which a curable biocompatible material is to be delivered, and at least one interior lumen disposed between the proximal and distal portions through which the biocompatible material is delivered; and
a confinement member adjacent the distal portion of the cannula having an unexpanded position and an expanded position defining an open cavity about the distal portion of the cannula, and the confinement member in fluid communication with the at least one interior lumen of the cannula and adapted to retain at least a portion of the biocompatible material.

2. The device of claim 1, wherein the confinement member is an inflatable member which is deflated when in the unexpanded position and is inflated when in the expanded position.

3. The device of claim 2, wherein the inflatable member is actuated in a manner selected from the group consisting of pneumatic, hydraulic or mechanical actuation.

4. The device of claim 1, wherein the confinement member is formed integrally with the cannula.

5. The device of claim 1, wherein the confinement member is cone shaped when in the expanded position.

6. The device of claim 1, wherein the confinement member includes a reinforcing structure to provide a desired shape to the confinement member when in the expanded position.

7. The device of claim 1, further comprising:
a vacuum source coupled to the at least one lumen for aspirating fluid from the cavity.

8. The device of claim 1, further comprising:
a source of curable biocompatible material coupled to the at least one lumen for supplying the biocompatible material to the cavity.

9. The device of claim 1, further comprising: a light source for photo initiating cross-linking of the curable biocompatible material.

10. The device of claim 9, wherein the light source is internal to the cannula.

11. The device of claim 9, wherein the light source is selected from the group consisting of a visible and an ultraviolet light source.

12. The device of claim 1, wherein at least a portion of the confinement member is a transparent material to provide visualization of the surgical site through the portion of the confinement member.

13. The device of claim 1, wherein at least a portion of the confinement member is a material capable of transmitting light therethrough for photo initiating cross-linking of the biocompatible material.

14. The device of claim 13, wherein the portion of the confinement member is capable of transmitting at least one of visible light or ultraviolet light therethrough.

15. The device of claim 13, wherein the confinement member is made of polyurethane.

16. A method of delivering a curable biocompatible material to a site in the body, comprising:
positioning a distal portion of a cannula adjacent the body site;
actuating a confinement member to define an open cavity about the distal portion of the cannula;
introducing the curable biocompatible material into the cavity through the cannula; and
initiating cross-linking of the curable biocompatible material at the body site.

17. The method of claim 16, wherein actuating comprises:
inflating the confinement member from an unexpanded position to an expanded position to define the cavity.

18. The method of claim 16, comprising:
deflating and removing from the surgical site the confinement member after the biocompatible material has at least partially cured.

19. The method of claim 16, comprising:
visualizing the surgical site through at least a portion of the confinement member.

20. The method of claim 16, wherein initiating cross-linking of the curable biocompatible material comprises:
initiating cross-linking by at least one of photo initiation, thermal initiation, or chemical initiation.

21. The method of claim 16, wherein initiating cross-linking of the curable biocompatible material comprises:
photo initiating the cross-linking of the biocompatible material with a source of at least one of visible or ultraviolet light.

22. The method of claim 21, wherein the light passes through at least a portion of the confinement member.

23. The method of claim 16, further comprising: applying vacuum pressure to aspirate fluid from the cavity prior to introducing the curable material.

24. The method of claim 16 for repairing cartilage within a joint.
